(19) United States
(12) Patent Application Publication
(10) Pub. No.: US 2013/0261536 A1
(43) Pub. Date: Oct. 3, 2013

(54) System and Method for Cholecystectomy

(71) Applicant: Joe D. Sartor, Longmont, CO (US)

(72) Inventor: Joe D. Sartor, Longmont, CO (US)

(73) Assignee: TYCO Healthcare Group LP, Boulder, CO (US)

(21) Appl. No.: 13/431,373

(22) Filed: Mar. 27, 2012

(51) Int. Cl. A61M 37/00; A61B 17/122

(52) U.S. Cl. 604/23

(57) ABSTRACT

A method for performing a cholecystectomy procedure is disclosed. The method includes the steps of ligating a cystic artery and a cystic duct of a gallbladder, separating the gallbladder from a liver by grasping the gallbladder and stretching a connective tissue therebetween and positioning a plasma applicator adjacent the connective tissue. The method also includes the steps of generating a selectively reactive plasma effluent at the plasma applicator and directing the selectively reactive plasma effluent at the connective tissue to separate the gallbladder from the liver.

### BACKGROUND

1. Technical Field

The present disclosure relates to plasma devices and processes for surface processing and tissue removal. More particularly, the disclosure relates to a system and method for generating and directing chemically reactive, plasma-generated species in a plasma device along with excited-state species (e.g., energetic photons) that are specific to the supplied feedstocks for treating tissue.

2. Background of Related Art

Electrical discharges in dense media, such as liquids and gases at or near atmospheric pressure, can, under appropriate conditions, result in plasma formation. Plasmas have the unique ability to create large amounts of chemical species, such as ions, radicals, electrons, excited-state (e.g., metastable) species, molecular fragments, photons, and the like. The plasma species may be generated in a variety of internal energy states or external kinetic energy distributions by tailoring plasma electron temperature and electron density. In addition, adjusting spatial, temporal and temperature properties of the plasma creates specific changes to the material being irradiated by the plasma species and associated photon fluxes. Plasmas are also capable of generating photons including energetic ultraviolet photons that have sufficient energy to initiate photochemical and photocatalytic reaction paths in biological and other materials that are irradiated by the plasma photons.

### SUMMARY

Plasmas have broad applicability to provide alternative solutions to industrial, scientific and medical needs, especially workpiece surface processing at low temperature. Plasmas may be delivered to a workpiece, thereby affecting multiple changes in the properties of materials upon which the plasmas impinge. Plasmas have the unique ability to create large fluxes of radiation (e.g., ultraviolet), ions, photons, electrons and other excited-state (e.g., metastable) species which are suitable for performing material property changes with high spatial, material selectivity, and temporal control. Plasmas may also remove a distinct upper layer of a workpiece but have little or no effect on a separate underlayer of the workpiece or it may be used, to selectively remove a particular tissue from a mixed tissue region or selectively remove a tissue with minimal effect to adjacent organs of different tissue type.

One suitable application of the unique chemical species is to drive non-equilibrium or selective chemical reactions at or within the workpiece to provide for selective removal of only certain types of materials. Such selective processes are especially sought in biological tissue processing (e.g., mixed or multi-layered tissue), which allows for cutting and removal of tissue at low temperatures with differential selectivity to underlayers and adjacent tissues. This is particularly useful for removal of biofilms, mixtures of fatty and muscle tissue, debridement of surface layers and removing of epoxy and other non-organic materials during implantation procedures.

The plasma species are capable of modifying the chemical nature of tissue surfaces by breaking chemical bonds, substituting or replacing surface-terminating species (e.g., surface functionalization) through volatilization, gasification or dissolution of surface materials (e.g., etching). With proper techniques, material choices and conditions, one can remove one type of tissue entirely without affecting a nearby different type of tissue. Controlling plasma conditions and parameters (including S-parameters, V, I, Θ, and the like) allows for the selection of a set of specific particles, which, in turn, allows for selection of chemical pathways for material removal or modification as well as selectivity of removal of desired tissue type. The present disclosure provides for a system and method for creating plasma under a broad range of conditions including tailored geometries, various plasma feedstock media, number and location of electrodes and electrical excitation parameters (e.g., voltage, current, phase, frequency, pulse condition, etc.).

The supply of electrical energy that ignites and sustains the plasma discharge is delivered through substantially conductive electrodes that are in contact with the ionizable media and other plasma feedstocks. The present disclosure also provides for methods and apparatus that utilize specific electrode structures that improve and enhance desirable aspects of plasma operation such as higher electron temperature and higher secondary emission. In particular, the present disclosure provides for porous media for controlled release of chemical reactants.

Controlling plasma conditions and parameters allows for selection of a set of specific particles, which, in turn, allows for selection of chemical pathways for material removal or modification as well as selectivity of removal of desired tissue type. The present disclosure also provides for a system and method for generating plasmas that operate at or near atmospheric pressure. The plasmas include electrons that drive reactions at material surfaces in concert with other plasma species. Electrons delivered to the material surface can initiate a variety of processes including bond scission, which enables volatilization in subsequent reactions. The electron-driven reactions act synergistically with associated fluxes to achieve removal rates of material greater than either of the reactions acting alone.

A method for performing a cholecystectomy procedure is contemplated by the present disclosure. The method includes the steps of ligating a cystic artery and a cystic duct of a gallbladder, separating the gallbladder from a liver by grasping the gallbladder and stretching a connective tissue therebetween and positioning a plasma applicator adjacent the connective tissue. The method also includes the steps of generating a selectively reactive plasma effluent at the plasma applicator and directing the selectively reactive plasma effluent at the connective tissue to separate the gallbladder from the liver.

The present disclosure also provides for another method for performing a cholecystectomy procedure. The method includes the steps of ligating a cystic artery and a cystic duct of a gallbladder, separating the gallbladder from a liver by grasping the gallbladder and stretching a connective tissue therebetween and positioning a plasma applicator adjacent the connective tissue. The plasma applicator includes a shaft having a proximal portion and a deflectable distal portion and a lumen defined therein terminating in an opening at a distal end of the distal portion, the lumen being in fluid communication with an ionizable media source. The applicator also includes a plurality of electrodes disposed at the distal portion and coupled to a power source. The method further includes the steps of generating a selectively reactive plasma effluent at the plasma applicator and directing the selectively reactive plasma effluent at the connective tissue to separate the gallbladder from the liver.

Another method for performing a cholecystectomy procedure is also contemplated by the present disclosure. The method includes the steps of ligating a cystic artery and a cystic duct of a gallbladder, separating the gallbladder from a liver by grasping the gallbladder and stretching a connective tissue therebetween and positioning a plasma applicator adjacent the connective tissue. The method also includes the steps of selecting at least one precursor feedstock having higher chemical reactivity with the connective tissue than with the gallbladder and the liver tissue, supplying ionizable media and the at least one precursor feedstock to the plasma applicator, igniting the ionizable media and the at least one precursor feedstock at the plasma applicator to form a selectively reactive plasma effluent and directing the selectively reactive plasma effluent at the connective tissue to separate the gallbladder from the liver.

### BRIEF DESCRIPTION OF THE DRAWINGS

The accompanying drawings, which are incorporated in and constitute a part of this specification, illustrate exemplary embodiments of the disclosure and, together with a general description of the disclosure given above, and the detailed description of the embodiments given below, serve to explain the principles of the disclosure, wherein:

FIG. 1 is a schematic diagram of a plasma system according to the present disclosure;

FIG. 2 is a schematic view of a plasma device according to the present disclosure;

FIG. 3 is a cross-sectional view of the plasma device of FIG. 2;

FIG. 4 is a perspective cross-section view of a distal portion of the plasma device according to an embodiment of the present disclosure;

FIG. 5 is a perspective cross-section view of a distal portion of the plasma device according to an embodiment of the present disclosure;

FIG. 6 is a perspective view of a surgical site; and

FIG. 7 is a flow chart of a method according to the present disclosure.

### DETAILED DESCRIPTION

Plasmas are generated using electrical energy that is delivered as either direct current (DC) electricity or alternating current (AC) electricity at frequencies from about 0.1 hertz (Hz) to about 100 gigahertz (GHz), including radio frequency (“RF”, from about 0.1 MHz to about 100 MHz) and microwave (“MW”, from about 0.1 GHz to about 100 GHz) bands, using appropriate generators, electrodes, and antennas. Choice of excitation frequency, the workpiece, as well as the electrical circuit that is used to deliver electrical energy to the circuit affects many properties and requirements of the plasma. The performance of the plasma chemical generation, the delivery system and the design of the electrical excitation circuitry are interrelated—as the choices of operating voltage, frequency and current levels (as well as phase) effect the electron temperature and electron density. Further, choices of electrical excitation and plasma device hardware also determine how a given plasma system responds dynamically to the introduction of new ingredients to the host plasma gas or liquid media. The corresponding dynamic adjustment of the electrical drive, such as via dynamic match networks or adjustments to voltage, current, or excitation frequency may be used to maintain controlled power transfer from the electrical circuit to the plasma.

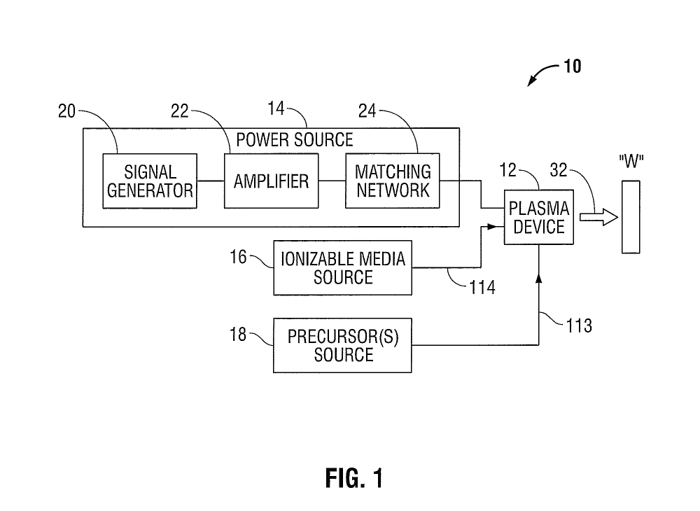

Referring initially to FIG. 1, a plasma system 10 is disclosed. The system 10 includes a plasma device 12 that is coupled to a power source 14, an ionizable media source 16 and a precursor source 18. Power source 14 includes any suitable components for delivering power or matching impedance to plasma device 12. More particularly, the power source 14 may be any radio frequency generator or other suitable power source capable of producing power to ignite the ionizable media to generate plasma. The plasma device 12 may be utilized as an electrosurgical pencil for application of plasma to tissue and the power source 14 may be an electrosurgical generator that is adapted to supply the device 12 with electrical power at a frequency from about 0.1 MHz to about 2,450 MHz and in another embodiment from about 1 MHz to about 13.56 MHz. The plasma may also be ignited by using continuous or pulsed direct current (DC) electrical energy.

The precursor source 18 may be a bubbler or a nebulizer configured to aerosolize precursor feedstocks prior to introduction thereof into the device 12. The precursor source 18 may also be a micro droplet or injector system capable of generating predetermined refined droplet volume of the precursor feedstock from about 1 femtoliter to about 1 nanoliter in volume. The precursor source 18 may also include a microfluidic device, a piezoelectric pump, or an ultrasonic vaporizer.

The system 10 provides a flow of plasma through the device 12 to a workpiece “W” (e.g., tissue). Plasma feedstocks, which include ionizable media and precursor feedstocks, are supplied by the ionizable media source 16 and the precursor source 18, respectively, to the plasma device 12. During operation, the precursor feedstock and the ionizable media are provided to the plasma device 12 where the plasma feedstocks are ignited to form plasma effluent containing ions, radicals, photons from the specific excited species and metastables that carry internal energy to drive desired chemical reactions in the workpiece “W” (e.g., tissue) or at the surface thereof. The feedstocks may be mixed upstream from the ignition point or midstream thereof (e.g., at the ignition point) of the plasma effluent, as shown in FIG. 1 and described in more detail below.

The ionizable media source 16 provides ionizable feedstock to the plasma device 12. The ionizable media source 16 is coupled to the plasma device 12 and may include a storage tank and a pump (not explicitly shown). The ionizable media may be a liquid or a gas such as argon, helium, neon, krypton, xenon, radon, carbon dioxide, nitrogen, hydrogen, oxygen, etc. and their mixtures, and the like, or a liquid. These and other gases may be initially in a liquid form that is gasified during application.

The precursor source 18 provides precursor feedstock to the plasma device 12. The precursor feedstock may be either in solid, gaseous or liquid form and may be mixed with the ionizable media in any state, such as solid, liquid (e.g., particulates or droplets), gas, and the combination thereof. The precursor source 18 may include a heater, such that if the precursor feedstock is liquid, it may be heated into gaseous state prior to mixing with the ionizable media.

In one embodiment, the precursors may be any chemical species capable of forming reactive species such as ions, electrons, excited-state (e.g., metastable) species, molecular fragments (e.g., radicals) and the like, when ignited by electrical energy from the power source 14 or when undergoing collisions with particles (electrons, photons, or other energy-bearing species of limited and selective chemical reactivity) formed from ionizable media 16. More specifically, the precursors may include various reactive functional groups, such as acyl halide, alcohol, aldehyde, alkane, alkene, amide, amine, butyl, carboxlic, cyanate, isocyanate, ester, ether, ethyl, halide, haloalkane, hydroxyl, ketone, methyl, nitrate, nitro, nitrile, nitrite, nitroso, peroxide, hydroperoxide, oxygen, hydrogen, nitrogen, and combination thereof. In embodiments, the chemical precursors may be water, halogenoalkanes, such as dichloromethane, tricholoromethane, carbon tetrachloride, difluoromethane, trifluoromethane, carbon tetrafluoride, and the like; peroxides, such as hydrogen peroxide, acetone peroxide, benzoyl peroxide, and the like; alcohols, such as methanol, ethanol, isopropanol, ethylene glycol, propylene glycol, alkalines such as NaOH, KOH, amines, alkyls, alkenes, and the like. Such chemical precursors may be applied in substantially pure, mixed, or soluble form.

The precursors and their functional groups may be delivered to a surface to react with the surface species (e.g., molecules) of the workpiece “W.” In other words, the functional groups may be used to modify or replace existing surface terminations of the workpiece “W.” The functional groups react readily with the surface species due to their high reactivity and the reactivity imparted thereto by the plasma. In addition, the functional groups are also reacted within the plasma volume prior to delivering the plasma volume to the workpiece.

Some functional groups generated in the plasma can be reacted in situ to synthesize materials that subsequently form a deposition upon the surface. This deposition may be used for stimulating healing, killing bacteria, and increasing hydrophilic or hydroscopic properties. In addition, deposition of certain function groups may also allow for encapsulation of the surface to achieve predetermined gas/liquid diffusion, e.g., allowing gas permeation but preventing liquid exchange, to bond or stimulate bonding of surfaces, or as a physically protective layer.

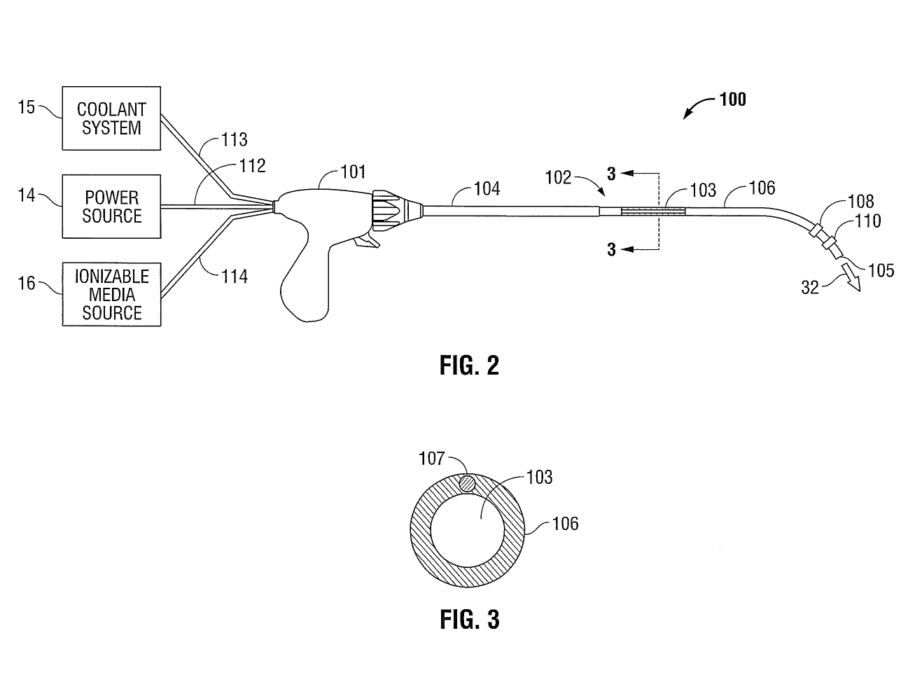

With reference to FIGS. 1 and 2, the precursor source 18 and the ionizable media source 16 may be coupled to the plasma device 12 via tubing 114 and 113, respectively. The tubing 114 and 113 may be combined into unified tubing to deliver a mixture of the ionizable media and the precursor feedstock to the device 12 at a proximal end thereof. This allows for the plasma feedstocks, e.g., the precursor feedstock and the ionizable gas, to be delivered to the plasma device 12 simultaneously prior to ignition of the mixture therein.

In another embodiment, the ionizable media source 16 and the precursors source 18 may be coupled to the plasma device 12 via the tubing 114 and 113 at separate connections, such that the mixing of the feedstocks occurs within the plasma device 12 upstream from the ignition point. In other words, the plasma feedstocks are mixed proximally of the ignition point, which may be any point between the respective sources 16 and 18 and the plasma device 12, prior to ignition of the plasma feedstocks to create the desired mix of the plasma effluent species for each specific surface treatment on the workpiece “W.”

In a further embodiment, the plasma feedstocks may be mixed midstream, e.g., at the ignition point or downstream of the plasma effluent, directly into the plasma. It is also envisioned that the ionizable media may be supplied to the device 12 proximally of the ignition point, while the precursor feedstocks are mixed therewith at the ignition point. In a further illustrative embodiment, the ionizable media may be ignited in an unmixed state and the precursors may be mixed directly into the ignited plasma. Prior to mixing, the plasma feedstocks may be ignited individually. The plasma feedstock is supplied at a predetermined pressure to create a flow of the medium through the device 12, which aids in the reaction of the plasma feedstocks and produces a plasma effluent. The plasma according to the present disclosure is generated at or near atmospheric pressure under normal atmospheric conditions.

The system 10 also includes a coolant system 15 for cooling the device 12 and particularly the plasma plume 32. The coolant system 15 includes a supply pump 17 and a supply tank 18. The supply pump 17 may be a peristaltic pump or any other suitable type of pump known in the art. The supply tank 17 stores the coolant fluid (e.g., saline) and, in one embodiment, may maintain the fluid at a predetermined temperature. In another embodiment, the coolant fluid may be a gas and/or a mixture of fluid and gas.

With reference to FIGS. 2 and 3, the device 12 is shown as a plasma applicator 100. The applicator 100 may be used in either open or endoscopic procedures. The applicator 100 includes a handle 101 and a longitudinal shaft 102 coupled thereto. The shaft 102 includes a proximal portion 104 coupled to the handle 101 and a distal portion 106. The catheter shaft 102 includes a plasma lumen 103 defined therein and extending the entire length thereof and terminating in an opening 105 at distal end of the distal portion 106. The shaft 102 may have a diameter from about 5 mm to about 10 mm allowing the applicator 100 to be inserted through operating ports for application of the plasma effluent 32 at the operating site during laparscopic procedures or through natural body orifices. In another embodiment, the applicator 100 may be configured for use within or accompanied by a flexible endoscope.

In one embodiment, the distal portion 106 is configured for controlled deflection. A pull-wire 107 (FIG. 3) or another suitable actuation mechanism extends from the handle 101 at the proximal end of the catheter 100 through a lumen in the catheter shaft 102 and is fastened to the distal portion 106. The pull-wire 107 is movable from a first generally relaxed position wherein the distal portion 106 is disposed in a generally longitudinally-aligned position relative to the proximal portion 104 to a second retracted or tensed position wherein the distal portion 106 flexes (e.g., deflects) from the proximal portion 104 at a desired angle as shown in FIG. 2.

The distal portion 106 is constructed to be more flexible than the proximal portion 104, such that when the handle 101 is pulled back or otherwise actuated, the pull-wire bends the distal portion 106 from an undeflected position to a deflected position. In particular, the proximal portion 104 may include a wire or other support materials (not shown) therein to provide tensile strength to the catheter shaft 102 while still maintaining flexibility for maneuvering through a vascular system. The distal portion 106 is formed from a flexible biocompatible material such as polytetrafluoroethylene, polyurethane, polyimide, and the like to allow for maneuverability thereof.

The applicator 100 includes two or more electrodes 108 and 110 disposed at the distal portion 106. The electrodes 108 and 110 may be formed from a conductive material and have a ring-like shape. The electrodes 108 and 110 may be disposed over the distal portion 106 to provide for capacitive coupling with the ionizable media. In another embodiment, the electrodes 108 and 110 may be formed as needle electrodes (e.g., pointed tip) and may be disposed within the distal portion 106.

The electrodes 108 and 110 are coupled to conductors (not shown) that extend through the catheter shaft 102 and are connected to the power source 14 via electrical connectors 112. The catheter shaft 102 is also coupled to the ionizable media source 16 via gas tubing 114 and to the precursors source 16 via tubing 113. The ionizable media source 16 and the precursors source 16 may include various flow sensors and controllers (e.g., valves, mass flow controllers, etc.) to control the flow of ionizable media to the applicator 100. In particular, the lumen 103 is in gaseous and/or liquid communication with the ionizable media source 16 and the precursors source 18 allowing for the flow of ionizable media and precursor feedstocks to flow through the catheter shaft 102 to the distal portion 106. The ionizable media in conjunction with the precursor feedstocks is ignited by application of energy through the electrodes 108 and 110 to form plasma plume 32 exiting through the opening 105.

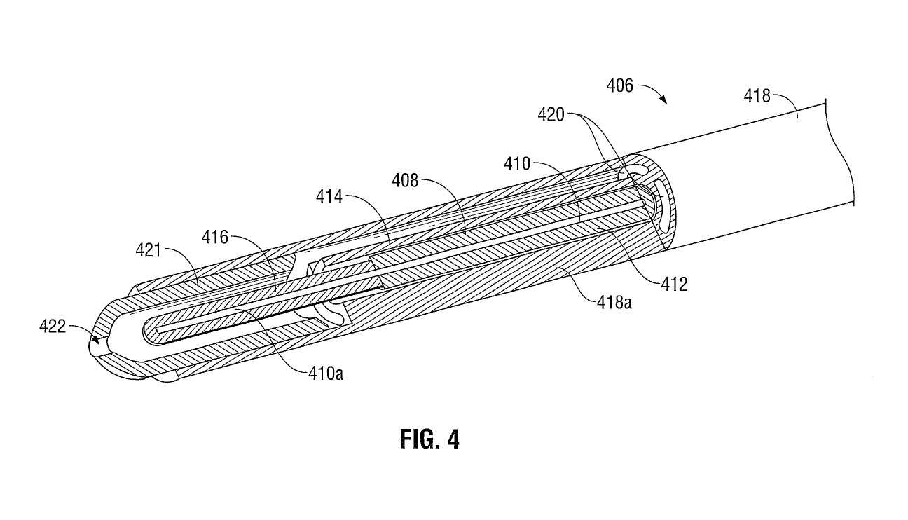

FIG. 4 illustrates another embodiment of a distal portion 406 of the applicator 100. The distal portion 406 includes an electrode assembly 408 having an inner electrode 410 surrounded by an insulator jacket 412. The electrode assembly 408 may optionally include an outer electrode 414 disposed over the insulator jacket 412 in a coaxial manner relative to the inner electrode 410. The inner and outer conductors 410 and 414 act as a first and second pole, respectively, and may be constructed of any conductive material including, but not limited to, copper, gold, stainless steel and the like. The metals may also be plated with other conductive materials, e.g., to improve conductivity or decrease energy loss, etc. In one embodiment, the inner and outer conductors 410 and 414 may be formed from a coaxial semi-rigid or flexible cable. In further embodiments, the inner conductor 410 may be coupled to or be integrally formed as the pull-wire 107.

The inner conductor 410 includes a distal conducting portion 410a extending distally past the end of the insulator jacket 412 and the outer electrode 414. The conducting portion 410a is enclosed in an inner cap 416. The inner cap 416 may have the same diameter as the insulator jacket 412 to provide for a smooth transition therebetween. In embodiments, the inner cap 416 may be made from either an insulating material or a conductive material. In embodiments in which the inner cap 416 is conductive, the cap 416 may include an insulating coating disposed thereon. The coating may be disposed on ionizable media contact surfaces of the cap 416. The inner cap 416 and the insulator jacket 412 may be integrally formed from any suitable dielectric material. The cap 416 may be formed from a conductive material having an insulative coating disposed on an outer surface thereof. In embodiments, the insulator jacket 412 may extend distally to enclose the distal conducting portion 410a, obviating the need for the inner cap 416.

The electrode assembly 408 is enclosed within a sheath 418, which in embodiments may be formed from the catheter shaft 102. The sheath 418 has tubular structure and may have any suitable cross-sectional shape (e.g., oval, circular, polygonal, etc.). The sheath 418 includes a proximal sheath portion 418a configured to secure the electrode assembly 408 (e.g., frictionally). The proximal sheath portion 418a defines one or more lumens 420 therein. The lumens 420 are in gaseous communication with the plasma lumen 103 allowing for the flow of ionizable media and precursor feedstocks to flow through the sheath 418 to the conducting portion 410a. The lumens 420 are disposed radially about the electrode assembly 408.

The distal portion 406 also includes an end cap 421 defining one or more apertures 422 therein. The distal end of the end cap 421 may have a hemispherical or funnel shape suitable for directing the plasma effluent out of the distal portion 406. The cross-sectional shape of the proximal end of the end cap 421 may substantially match the cross-sectional shape of the distal end of the sheath 418. The end cap 421 may couple either on the outside or inside the sheath 418. In embodiments, the end cap 421 may be made from either an insulating material or a conductive material. In embodiments in which the end cap 421 is conductive, the end cap 421 may be in electro-mechanical contact with the outer conductor 414, thereby allowing the end cap 421 to be used as a second pole the end cap 421. In further embodiments, the end cap 421 may include an insulating coating disposed thereon. The coating may be disposed on ionizable media contact surfaces of the end cap 421.

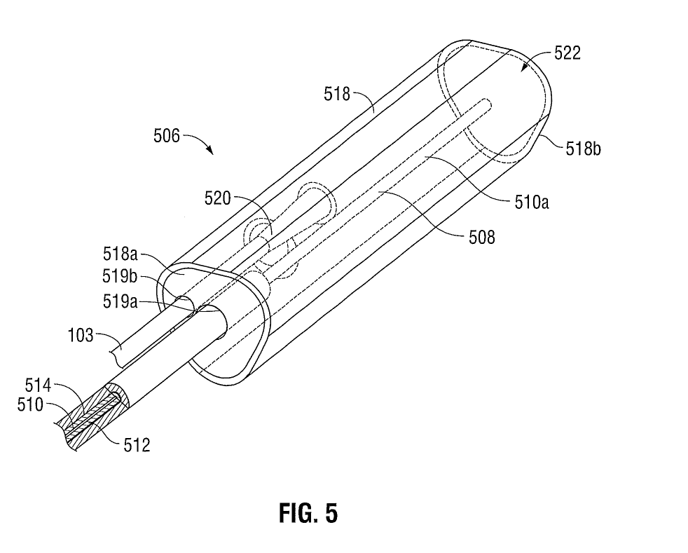

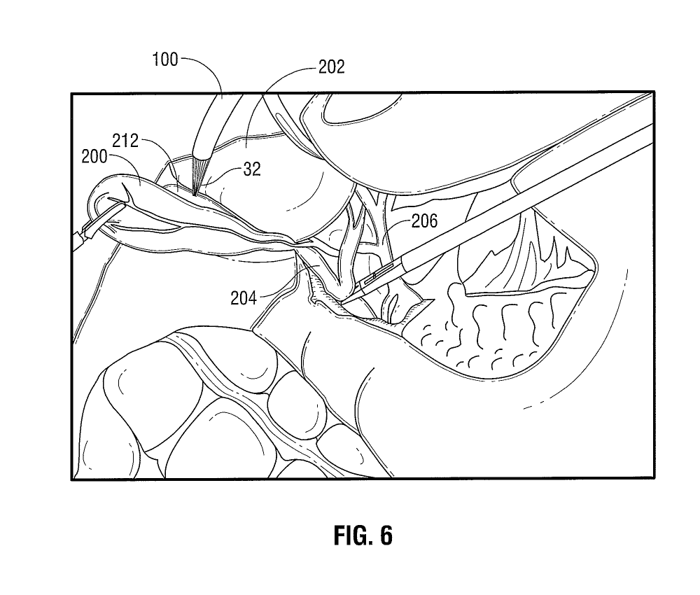

FIG. 5 illustrates another embodiment of a proximal handle portion 506 of the applicator 100. The distal portion 506 includes an electrode assembly 508 having an inner electrode 510 surrounded by an insulator jacket 512. The electrode assembly 508 may optionally include an outer electrode 514 disposed over the insulator jacket 512 in a coaxial manner relative to the inner electrode 510. The inner and outer conductors 510 and 514 act as a first and second pole, respectively, and may be constructed of any suitable conducting materials including, but not limited to, copper, gold, stainless steel and the like. The metals may also be plated with other conductive materials, e.g., to improve conductivity or decrease energy loss, etc. In one embodiment, the inner and outer conductors 510 and 514 may be formed from a coaxial semi-rigid or flexible cable. In further embodiments, the inner conductor 510 may be coupled to or be integrally formed as the pull-wire 107.

The inner conductor 510 includes a distal conducting portion 510a extending distally past the end of the insulator jacket 512 and the outer electrode 514. The conducting portion 510a may be enclosed in an insulating cap, similar to the inner cap 416.

The electrode assembly 508 is enclosed within a handle 518, which in embodiments may be formed from the catheter shaft 102. The handle 518 has tubular granular (e.g., triangular cross-section) structure but may have any suitable cross-sectional shape (e.g., oval, circular, polygonal, etc.). The handle 518 may be formed from a dielectric material.

The handle 518 includes a distal end portion 518b defining one or more apertures 522 therein and a proximal end portion 518a configured to secure the electrode assembly 508 (e.g., frictionally). The proximal end portion 518a defines one or more openings 519a and 519b with the electrode assembly 508 being fitted through the opening 519a. The distal portion 506 also includes an inlet 520 fitted through the opening 519b. The inlet 520 is in gaseous communication with the plasma lumen 103 allowing for the flow of ionizable media and precursor feedstocks to flow through the funnel 518 to the conducting portion 510a.

The applicator 100 is suitable for either open or laparoscopic cholecystectomy procedures. During open cholecysectomies, the gallbladder is removed through incisions formed through the ribcage and/or abdominal muscles. In laparoscopic cholecysectomies, three or more small incisions are formed to allow for the insertion of operating ports therethrough. The operating ports provide for supply of insufflation gas to inflate the abdominal cavity and for insertion of various viewing and lighting instruments are used to observe the operation site.

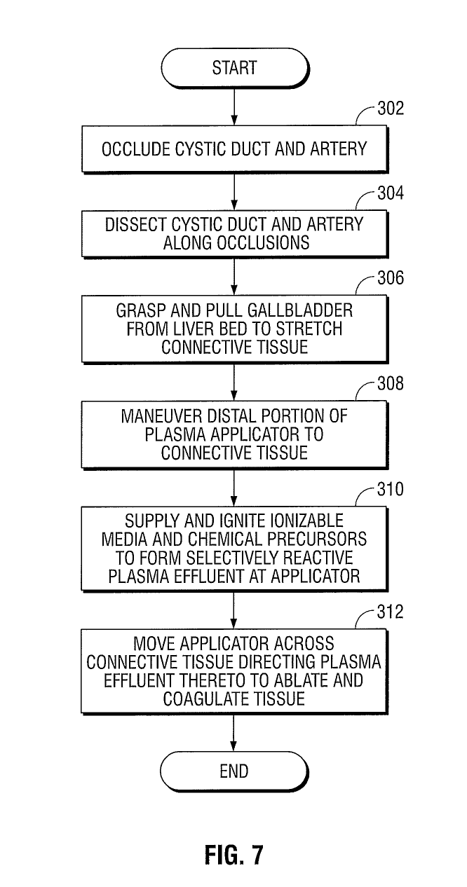

With reference to FIGS. 6 and 7, a cholecystectomy procedure using the applicator 100 is discussed. FIG. 6 illustrates the surgical site and FIG. 7 illustrates a flow chart of a method for removing the gallbladder. The surgical site includes a gallbladder 200 having a cystic duct 204 and a cystic artery 206. The gallbladder 200 is attached via connective tissue 212 (e.g., venous plexus) to a liver 202. In step 302, the cystic duct and artery 204 and 206 are occluded. This may be accomplished via application of surgical clips at two points using a clip applier. The cystic duct and artery 204 and 206 are then severed between the clips in step 304.

In another embodiment, the cystic duct and artery 204 and 206 may be ligated using electrosurgical sealing forceps. The forceps for clamping and sealing tissue includes first and second jaw members pivotally attached in opposing relation relative to one another that are movable from a first open position wherein the jaw members are disposed in spaced relation relative to one another to a second clamping position wherein the jaw members cooperate to grasp tissue therebetween. A drive rod assembly connects each of the jaw members to a source of electrical energy such that the jaw members are capable of conducting bipolar energy through the tissue held therebetween. A handle attached to the drive rod assembly imparts movement of the first and second jaw members from the first and second positions. The forceps seal tissue due to simultaneous application of pressure and electrosurgical energy by the jaw members to soften the vessel tissue and form a seal from the fused mass that occludes the lumen. Thereafter, the vessel is severed by cutting through the seal. One embodiment of the electrosurgical forceps is disclosed in a commonly-owned U.S. Pat. No. 6,458,130, entitled “Endoscopic Bipolar Electrosurgical Forceps,” the entire contents of which is incorporated by reference herein.

Once the cystic duct and artery 204 and 206 are ligated, in step 306, the gallbladder 200 is grasped via graspers 210 to pull the gallbladder 200 from the liver 202 to stretch the connective tissue 212. In step 308, the applicator 100 is brought adjacent the connective tissue 212. The distal portion 106 may be deflected to direct the plasma effluent 32 toward the connective tissue 212. In one embodiment, the deflection may be from about 0° to about 45° with respect to a longitudinal axis defined by the shaft 102. In step 310, the ionizable media along with precursors is supplied to the applicator 100 and is ignited therein to form the plasma effluent 32. In step 312, the applicator 100 is moved across the connective tissue 212 ensuring that the plasma effluent 32 is directed between the gallbladder 200 and the liver 202. The plasma effluent 32 effectively ablates connective tissue 212 while simultaneously cauterizing the venous plexus that exists therein.

The precursors supplied to the applicator 100 are specifically chosen to generate a selectively reactive plasma effluent 32. In other words, the precursors, when ignited, produce a plasma effluent 32 that interacts with certain types of tissue, namely connective tissue 212, and has little to no effect on the surrounding organs, namely, the gallbladder 200 and the liver 202. The selected precursor feedstocks have higher chemical reactivity with the connective tissue relative to the chemical reactivity with the gallbladder and the liver tissue. This allows for the plasma effluent 32 to sever the connective tissue 212 without damaging the surrounding organs. The plasma effluent 32 ablates and coagulates tissue via heat to stop bleeding at the edges thereof. Once the connective tissue 212 is dissected, the gallbladder 202 is separated from the liver bed and is extracted from the abdominal cavity. In embodiments, suitable precursors include, but are not limited to, halogen containing compounds such as halogenoalkanes, e.g., dichloromethane, tricholoromethane, carbon tetrachloride, difluoromethane, trifluoromethane, carbon tetrafluoride, and combinations thereof; peroxides, such as hydrogen peroxide, acetone peroxide, benzoyl peroxide, and combinations thereof. Suitable ionizable media include any of the gases or liquids discussed above.

In conventional laparoscopic cholecystectomies, the gallbladder is lifted ventrally and dissected typically with an electrosurgical electrode. Maintaining the electrosurgical effect of the electrode at the connective tissue is challenging and is dependent on the tension applied to the gallbladder. The tension on the connective tissue reduces its density, thereby making the tissue easier to cut with any energy source, however, typical electrosurgical devices are prone to direct the current to one of the organs on either side of the connective tissue plane. Failure to stay within the connective tissue results in rupture and subsequent spilling of the contents of the gall bladder, or an unintentional cut to the liver bed, which causes excessive bleeding.

The applicator 100, being a non-current applying device, chases the gap between the two organs with the plasma effluent 32 where the tissue is less dense and is more easily affected by the chemical and thermodynamic properties of the plasma. The dissection of the connective tissue 212 is primarily due to volatilization by hydrothermal properties of the plasma effluent 32, which results in the contraction of the connective tissue 212 toward either opposing surface and out of the path of the plasma effluent 32.

As minimally invasive techniques progress toward procedures using a single port, the ability of the plasma applicator 100 to selectively direct its effect to the tissue enables higher efficacy and surgical speed. Further, the ability to direct the plasma jet off axis to the tissue via a fixed bend or flexure of the distal portion 106 facilitates application of the directionality of the plasma effluent 32.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure. In particular, as discussed above this allows the tailoring of the relative populations of plasma species to meet needs for the specific process desired on the workpiece surface or in the volume of the reactive plasma.

What is claimed is:

1. A method for performing a cholecystectomy procedure, the method comprising the steps of:
ligating a cystic artery and a cystic duct of a gallbladder;
separating the gallbladder from a liver by grasping the gallbladder and stretching a connective tissue therebetween;
positioning a plasma applicator adjacent the connective tissue;
generating a selectively reactive plasma effluent at the plasma applicator; and
directing the selectively reactive plasma effluent at the connective tissue to separate the gallbladder from the liver.

2. A method according to claim 1, wherein the ligating step further includes the steps of:
applying a plurality of clips onto the cystic artery and the cystic duct; and
severing the cystic artery and the cystic duct between at least two clips.

3. A method according to claim 1, wherein the ligating step further includes the steps of:
electrosurgically sealing the cystic artery and the cystic duct to form a seal therein; and
severing the cystic artery and the cystic duct between at least two clips.

4. A method according to claim 1, wherein the positioning step further includes the step of:
deflecting a distal portion of the plasma applicator to direct the distal portion toward the connective tissue.

5. A method according to claim 1, wherein the generating step further includes the steps of:
supplying ionizable media and at least one precursor feedstock to the plasma applicator; and
igniting the ionizable media and the at least one precursor feedstock at the plasma applicator to form the selectively reactive plasma effluent, wherein the ionizable media is argon.

6. A method according to claim 5, wherein the generating step further includes the step of:
selecting the at least one precursor feedstock having higher chemical reactivity with the connective tissue than with the gallbladder and the liver tissue, wherein the at least one precursor feedstock is hydrogen peroxide.

7. A method for performing a cholecystectomy procedure, the method comprising the steps of:
ligating a cystic artery and a cystic duct of a gallbladder;
separating the gallbladder from a liver by grasping the gallbladder and stretching a connective tissue therebetween;
positioning a plasma applicator adjacent the connective tissue, the plasma applicator including:
a shaft having a proximal portion and a deflectable distal portion and a lumen defined therein terminating in an opening at a distal end of the distal portion, the lumen being in fluid communication with an ionizable media source; and
a plurality of electrodes disposed at the distal portion and coupled to a power source;
generating a selectively reactive plasma effluent at the plasma applicator; and
directing the selectively reactive plasma effluent at the connective tissue to separate the gallbladder from the liver.

8. A method according to claim 7, wherein the ligating step further includes the steps of:
applying a plurality of clips onto the cystic artery and the cystic duct; and
severing the cystic artery and the cystic duct between at least two clips.

9. A method according to claim 7, wherein the ligating step further includes the steps of:
electrosurgically sealing the cystic artery and the cystic duct to form a seal therein; and
severing the cystic artery and the cystic duct between at least two clips.

10. A method according to claim 7, wherein the positioning step further includes the step of:
deflecting a distal portion of the plasma applicator to direct the distal portion toward the connective tissue.

11. A method according to claim 7, wherein the generating step further includes the steps of:
supplying ionizable media and at least one precursor feedstock to the plasma applicator; and
igniting the ionizable media and the at least one precursor feedstock at the plasma applicator to form the selectively reactive plasma effluent.

12. A method according to claim 11, wherein the generating step further includes the step of:
selecting the at least one precursor feedstock having higher chemical reactivity with the connective tissue than with the gallbladder and the liver tissue, wherein the at least one precursor feedstock is hydrogen peroxide.

13. A method for performing a cholecystectomy procedure, the method comprising the steps of:
ligating a cystic artery and a cystic duct of a gallbladder;
separating the gallbladder from a liver by grasping the gallbladder and stretching a connective tissue therebetween;
positioning a plasma applicator adjacent the connective tissue;
selecting at least one precursor feedstock having higher chemical reactivity with the connective tissue than with the gallbladder and the liver tissue;
supplying ionizable media and the at least one precursor feedstock to the plasma applicator;
igniting the ionizable media and the at least one precursor feedstock at the plasma applicator to form a selectively reactive plasma effluent; and
directing the selectively reactive plasma effluent at the connective tissue to separate the gallbladder from the liver.

14. A method according to claim 13, wherein the ligating step further includes the steps of:
applying a plurality of clips onto the cystic artery and the cystic duct; and
severing the cystic artery and the cystic duct between at least two clips.

15. A method according to claim 13, wherein the ligating step further includes the steps of:
electrosurgically sealing the cystic artery and the cystic duct to form a seal therein; and
severing the cystic artery and the cystic duct between at least two clips.

16. A method according to claim 13, wherein the positioning step further includes the step of:
deflecting a distal portion of the plasma applicator to direct the distal portion toward the connective tissue.

17. A medical device for treating tissue, the device comprising:
a housing having a substantially tubular shape and defining at least one lumen therethrough, the lumen in fluid communication with an ionizable media source configured to supply ionizable media thereto;
an inner electrode disposed within the housing; and
an outer electrode coaxially disposed around the inner electrode, wherein the inner and outer electrodes are coupled to a power source that energizes the inner and outer electrodes to ignite the ionizable media to form a plasma plume for treating tissue.

18. The medical device according to claim 17, further comprising an insulator jacket disposed between the inner and outer electrodes.

19. The medical device according to claim 17, further comprising an inner cap disposed over a distal end of the inner conductor, wherein the distal end of the inner conductor extends distally past a distal end of the outer conductor.

20. The medical device according to claim 19, further comprising an end cap having a substantially hemispherical shape, the end cap coupled to the housing and comprising at least one aperture defined therethrough.

21. The medical device according to claim 20, wherein the inner cap is formed from an electrically-insulating material and the end cap is formed from an electrically-conductive material.

22. The medical device according to claim 20, wherein the inner cap is formed from an electrically-conductive material and the end cap is formed from an electrically-insulating material.

23. The medical device according to claim 20, wherein the inner cap and the end cap include an electrically-insulating coating disposed on ionizable media contact surfaces.
